(19) United States
(12) Patent Application Publication
(10) Pub. No.: US 2013/0046192 A1
(43) Pub. Date: Feb. 21, 2013

(54) IMAGE-BASED PWV MEASUREMENT DEVICE AND METHOD

(71) Applicants: Yue-Der LIN, Taichung City (TW); Ching Che TSAI, Taichung City (TW); Hu Ying HO, Taoyuan County (TW); Shih Fan WANG, Yunlin County (TW); Kang Ping LIN, Taoyuan County (TW); Hen Hong CHANG, Taoyuan County (TW); Yung Ching CHANG, Taichung City (TW)

(72) Inventors: Yue-Der LIN, Taichung City (TW); Ching Che TSAI, Taichung City (TW); Hu Ying HO, Taoyuan County (TW); Shih Fan WANG, Yunlin County (TW); Kang Ping LIN, Taoyuan County (TW); Hen Hong CHANG, Taoyuan County (TW); Yung Ching CHANG, Taichung City (TW)

(21) Appl. No.: 13/307,883

(22) Filed: Nov. 30, 2011

(30) Foreign Application Priority Data
Aug. 19, 2011 (TW) 100129821

(51) Int. Cl. A61B 5/02

(52) U.S. Cl. 600/500

(57) ABSTRACT

An image-based PWV measurement device and method are provided. The measurement device comprises at least two light emitting units respectively projecting light beams to at least two detected regions on body surface; at least two light transmitting units respectively receiving and transmitting light signals measured at the different detected regions; an image sensing unit converting the light signals measured at the detected regions into image signals; a length measurement unit used to measure the distance between the detected regions; and an image analysis unit analyzing the image signals to obtain PPG signals for the detected regions. According to the PPG signals, the image analysis unit calculates the physiological parameters, including the perfusion index, respiration rate, pulse rate, stiffness index, reflection index, and PWV between the detected regions, which is derived according to the distance and the pulse transit time from the PPG signals of the two detected regions.

### BACKGROUND OF THE INVENTION

1. Field of the Invention

The present invention relates to a pulse wave velocity (PWV) measurement device and method, particularly to an image-based PWV measurement device and method, which uses image sensing elements to analyze the PPG (photoplethysmography) signals and obtain perfusion index, respiration rate, pulse rate, stiffness index, reflection index, and PWV.

2. Description of the Related Art

Science development is to improve the technology and quality of medicine. More and more physiological parameters such as electrocardiogram (ECG), blood pressure, body temperature, and blood oxygen concentration are used to monitor the patient in clinical medicine. Recently, nerrous activities have also been used in surgery. For example, AEP (Auditory Evoked Potential) and EEG (electroencephalogram)-based BIS (bispectral index) are used to evaluate the anesthetic depth induced by an anesthetic drug. The abovementioned measures enable the clinical doctors to grasp the physiological status of the patient more effectively.

In addition, the measurement of pulse wave velocity (PWV) is now adopted as an effective approach to evaluate the arterial stiffness in a non-invasive way.

The existing PWV measurement technology includes the method by ultrasound Doppler, the method by blood pressure, and the method by optical technique. The equipments for the method of ultrasound Doppler are usually very expensive and the experienced operator is required for such method as it is expected to align the ultrasound probe on the detected artery precisely. The method by blood pressure may have distorted waveform if the pressure sensor is not appropriately placed on the artery to be detected. The optical method, which uses photodiodes to detect optical signals, is restricted to be applied only to specific region of human body where the artery is close to body surface and the optical probe could be mounted. All the abovementioned methods for PWV measurement have their own respective problems to overcome.

Besides, the PWV measurement equipment and the transducer have to match well on the tested region. For example, the transducer for measuring PWV of the carotid artery is expensive and needs to be operated by well-trained technicians. When the transducer is replaced by one having a different specification, or when the measurement equipment is applied to another region, hardware incompatibility may occur.

The sensors for PWV measurement are normally expensive and lack universal adaptability. Different PWV measurement systems respectively need sensors of different specifications, thereby increasing the price and cost of PWV measurement.

Therefore, the persons skilled in the art are eager to develop a PWV measurement system and method that is able to effectively overcome the abovementioned problems.

### SUMMARY OF THE INVENTION

The primary objective of the present invention is to provide an image-based PWV measurement device and method, which adopts popular image sensing elements to receive the optical signals from several regions of a body, and which has flexibility to measure PWV from various regions of a body.

Another objective of the present invention is to provide an image-based PWV measurement device and method, which uses the distance between two measurement regions and the PPG signals received from the two measurement regions to work out PWV for arterial stiffness evaluation.

A further objective of the present invention is to provide an image-based PWV measurement device and method, which uses the PPG signals recorded in the image-processing device to obtain the perfusion indexes (PI) at different area, respiration rate, pulse rate, stiffness index (SI), and reflection index (RI).

To achieve the abovementioned objectives, the present invention proposes an image-based PWV measurement device, which comprises at least two light emitting units, at least two light transmitting units, an image sensing unit, a length measurement unit, and an image analysis unit. The light emitting units project light beams to at least two regions on body surface. The light transmitting units receive and transmit light signals measured at the detected regions. The image sensing unit is arranged corresponding to the light transmitting units and is used to convert the light signals which are transmitted from the detected regions into image signals. The length measurement unit is used to measure the distance between the two detected regions. The image analysis unit connects with the image sensing unit and the length measurement unit, and is used to analyze the image signals to obtain the PPG signals for the detected regions. The image analysis unit calculates the PWV between the two detected regions according to the distance and the pulse transit time (PTT) from the PPG signals of the two detected regions.

The present invention also proposes an image-based PWV measurement method, which comprises the following steps: providing the signal detection at least two detected regions and measuring the distance between the two detected regions; providing at least two light emitting units respectively projecting light beams to the two detected regions; receiving and transmitting the light signals measured at the two detected regions and converting the light signals into image signals; analyzing the image signals to obtain the PPG signals for the two detected regions; and calculating PWV between the two detected regions according to the distance and the pulse transit time (PTT) from the PPG signals of the two detected regions.

Below, the embodiments are described in detail in cooperation with the attached drawings to make the objectives, technical contents, characteristics and accomplishments of the present invention easily understood.

### BRIEF DESCRIPTION OF THE DRAWINGS

FIG. 1 is a block diagram schematically showing an image-based PWV measurement device according to one embodiment of the present invention;

FIG. 2 shows a flowchart of an image-based PWV measurement method according to one embodiment of the present invention;

FIG. 3A and FIG. 3B are block diagrams schematically showing light emitting units according to one embodiment of the present invention;

FIG. 4 shows a waveform of PPG diagram in time domain according to one embodiment of the present invention;

FIG. 5 is a block diagram schematically showing an image-based PWV measurement device according to another embodiment of the present invention;

FIG. 6 shows a flowchart of a parametric algorithm of a data processing unit according to one embodiment of the present invention; and

FIG. 7 shows PPG signals and a method to obtain the features thereof according to one embodiment of the present invention.

### DETAILED DESCRIPTION OF THE INVENTION

The present invention proposes an image-based PWV measurement device and method, wherein light emitting units project light beams to at least two detected regions, and wherein an image analysis unit records the PPG signals respectively from the tested regions and calculates PWV between the tested regions.

The image-based PWV measurement device and method of the present invention adopts popular image sensing elements to receive optical signals from several regions of a body and thus has flexibility to measure PWV from various regions of a body.

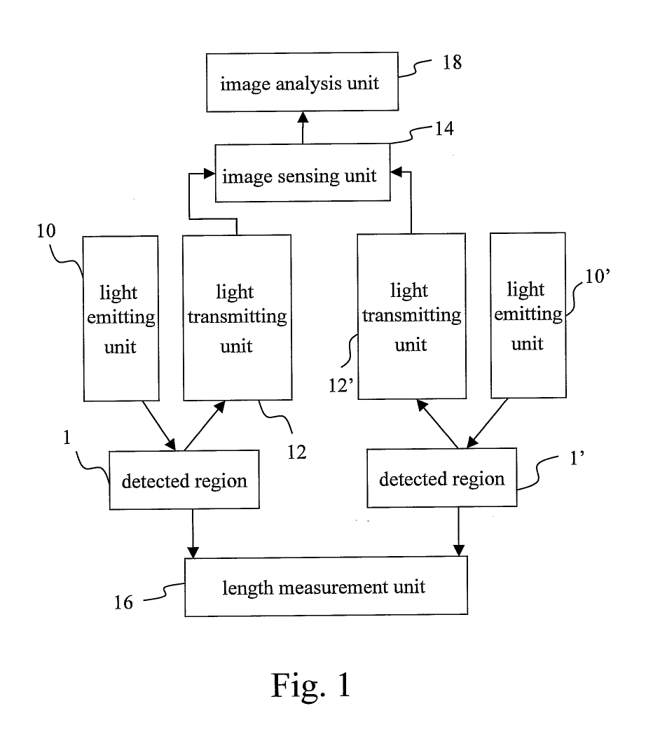

Refer to FIG. 1, which is a block diagram schematically showing an image-based PWV measurement device according to one embodiment of the present invention. The image-based PWV measurement device of the present invention is used to measure PWV between two detected regions 1 and 1′. The image-based PWV measurement device of the present invention comprises at least two light emitting units 10 and 10′, at least two light transmitting units 12 and 12′, an image sensing unit 14, a length measurement unit 16, and an image analysis unit 18. The light emitting units 10 and 10′ respectively project light beams to the detected regions 1 and F. The light transmitting units 12 and 12′ respectively receive and transmit light signals measured at the detected regions 1 and 1′. The image sensing unit 14 is arranged corresponding to the light transmitting units 12 and 12′ and is used to convert the light signals which are transmitted from the detected regions 1 and 1′ into image signals. The image analysis unit 18 connects with the image sensing unit 14 and the length measurement unit 16, and is used to analyze the image signals to obtain the PPG signals for the detected regions 1 and 1′.

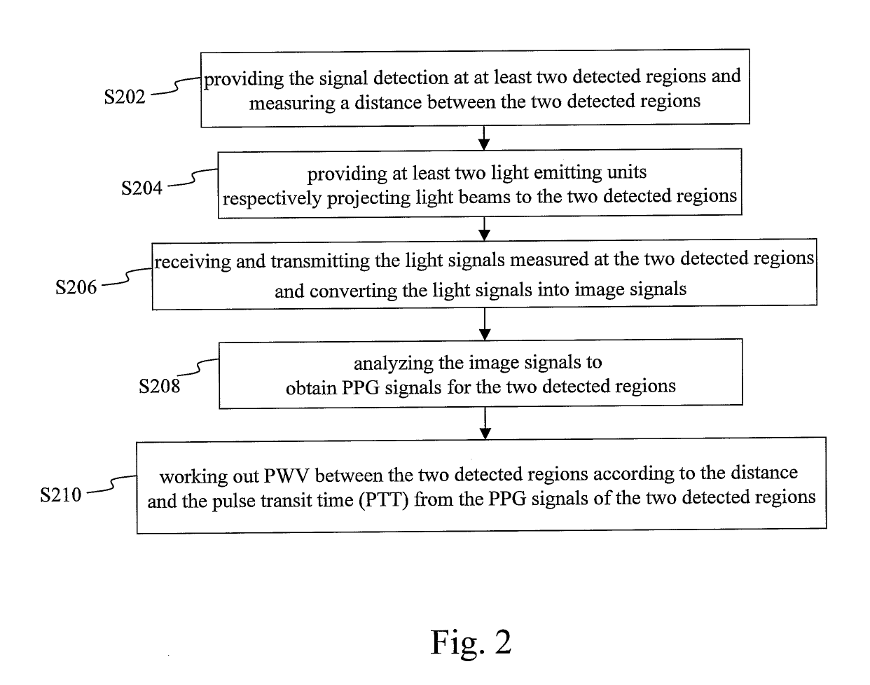

FIG. 2 shows a flowchart of an image-based PWV measurement method according to one embodiment of the present invention. Refer to FIG. 1 and FIG. 2 at the same time. The image-based PWV measurement method of the present invention is described in detail as following.

In Step S202, provide the signal detection at least two detected regions 1 and 1′, and use a length measurement unit 16 to measure the distance between the detected regions 1 and 1′.

In this embodiment, the carotid artery and the tip of the forefinger are respectively used to exemplify the detected regions 1 and 1′. However, this embodiment is only to exemplify the present invention but not to limit the scope of the present invention. The present invention doses not restrict that the detected regions should be the carotid artery and the tip of the forefinger. In practical applications, the user can determine the regions to be tested by himself.

In one embodiment, the length measurement unit 16 is a measuring tape for measuring the distance between the detected regions. However, the present invention does not restrict that the length measuring unit 16 should be a measuring tape. The length measuring unit 16 may be another type of length meter in other embodiments.

Next, in Step S204, provide at least two light emitting units 10 and 10′ respectively projecting light beams to the detected regions 1 and 1′.

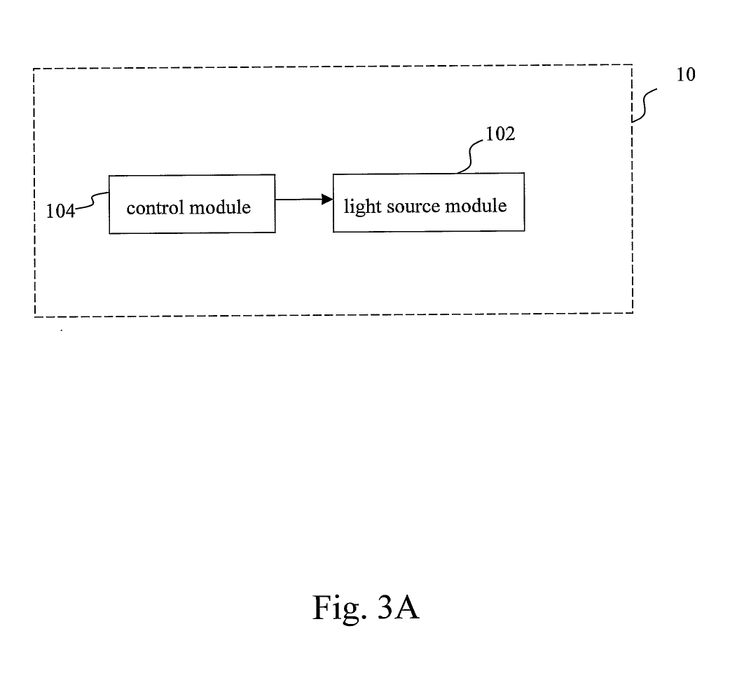

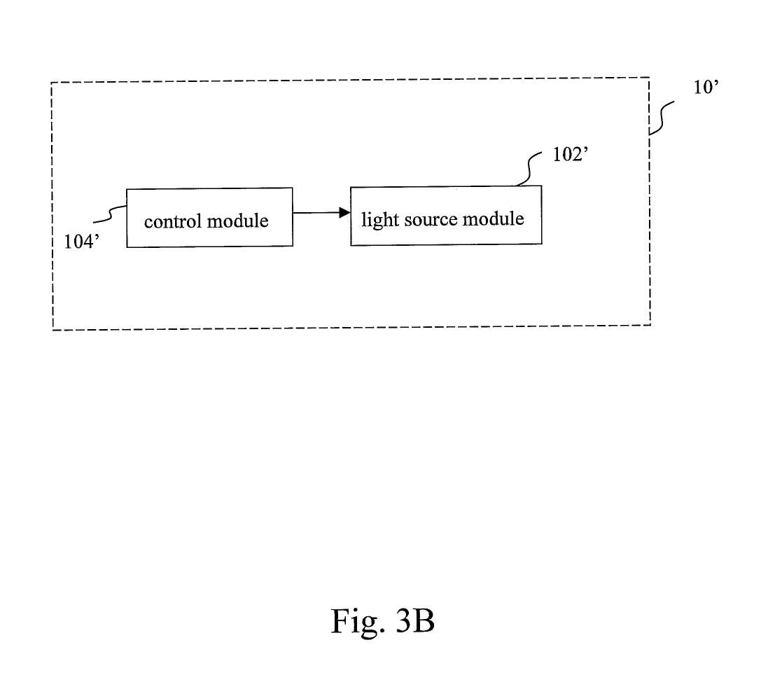

Refer to FIG. 3A and FIG. 3B. The light emitting units 10 and 10′ respectively comprise light source modules 102,102′ and control modules 104,104′. The light source modules 102 and 102′ respectively project light beams to the detected regions 1 and 1′. The control modules 104 and 104′ control the light source modules 102 and 102′ to respectively project light beams of different intensities for being emitted to different detected regions.

For example, each of the light source modules 102 and 102′ can be a light source emitting a monochromatic or multi-wavelength light beam, such as a light emitting diode (LED), a laser diode, or an incandescent lamp.

Next, in Step S206, the light transmitting units 12 and 12′ respectively receive and transmit the light signals measured at the detected regions 1 and 1′. In the present invention, the light signals measured at the detected regions 1 and 1′ may be the light signals reflected from the detected regions 1 and 1′ or the light signals transmitted through the detected regions 1 and 1′. In the present invention, each of the light transmitting units 12 and 12′ is a light conduction element such as optical fiber, a reflector, or a refractor that is free from external optical interference and is able to transmit a multi-wavelength light signal.

The light transmitting units 12 and 12′ send the light signals to the image sensing unit 14, and the image sensing unit 14 converts the light signals into image signals.

In the present invention, the image sensing unit 14 may be a CCD-based or CMOS-based digital camera device, wherein CCD and CMOS are respectively the abbreviations of “charge coupled device” and “complementary metal oxide semiconductor”. The image sensing unit 14 can record one or more images and can adjust the aperture, focal length, resolution, exposure rate and white balance in situ. The image sensing unit 14 can selectively transmit the images to the image processing device and present the images on a display device in realtime.

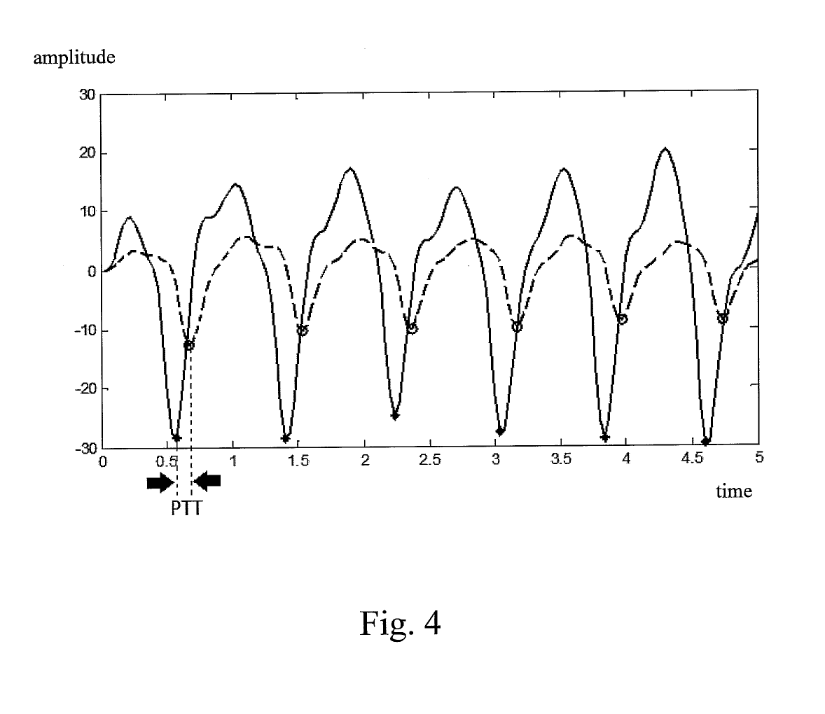

Next, in Step S208, the image analysis unit 18 analyzes the image signals captured by the image sensing unit 14 and displays the waveform of light intensity variation (shown in FIG. 4), whereby to obtain the PPG signals for the detected regions 1 and 1′.

Briefly speaking, the PPG signal is derived from the variation of the optical energy received by an optical sensor. In FIG. 4, the waveform designated by a solid curve is the PPG signal of the carotid artery; the waveform designated by a dotted curve is the PPG signal of the forefinger.

In Step S210, the image analysis unit 18 performs computation to obtain PWV between the detected regions 1 and 1′ according to the distance between the detected regions 1 and 1′ (obtained by the length measurement unit 16) and the abovementioned two PPG signals.

In detail, first, the image analysis unit 18 finds out the pulse transit time (PTT) between the two PPG signals in FIG. 4. Then, PTT is substituted into the equation:

PWV=distance/PTT

wherein “distance” is the distance between the detected regions 1 and 1′, and PTT is the pulse transit time. Thus the PWV between the detected regions 1 and 1′ is obtained.

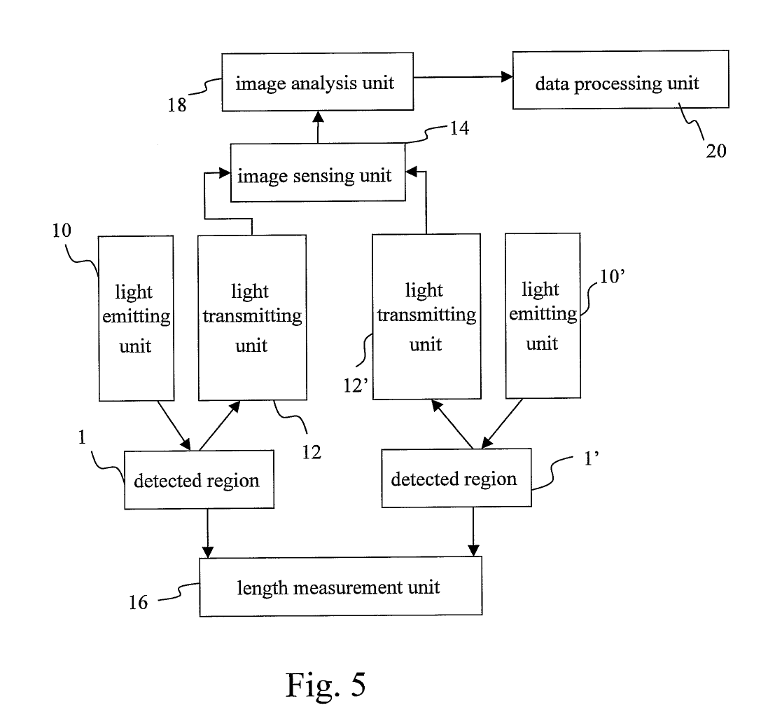

Refer to FIG. 5, which is a diagram schematically showing an image-based PWV measurement device according to another embodiment of the present invention. In this embodiment, the image-based PWV measurement device further comprises a data processing unit 20 connected with the image analysis unit 18, in addition to the light emitting units 10 and 10′, light transmitting units 12 and 12′, image sensing unit 14, length measurement unit 16, and image analysis unit 18.

In one embodiment, the data processing unit 20 can be a computer, a personal digital assistant, or a mobile phone. The data processing unit 20 analyzes the PPG signals to obtain physiological parameters, such as the perfusion index, respiration rate, pulse rate, stiffness index, reflection index and PWV.

The conventional non-invasive vessel-related measurement devices suffer from high cost and low flexibility because they have to use a unique or specified sensor as well as the front-end sensing circuit. However, the data processing unit 20 can use the built-in parametric algorithm to perform an image feature analysis and a filtering process on the PPG signals to work out the physiological parameters.

In detail, the image sensing unit 14 and the image analysis unit 18 capture the image. Next, the user designates the region of interest (ROI) in the human-machine interface (HMI) of the data processing unit 20. Next, the data processing unit 20 uses the parametric algorithm to undertake signal processing and parametric calculation and then presents the results on the human-machine interface. Thus a software process is completed. The software process will be executed repeatedly if another analysis or computation is required.

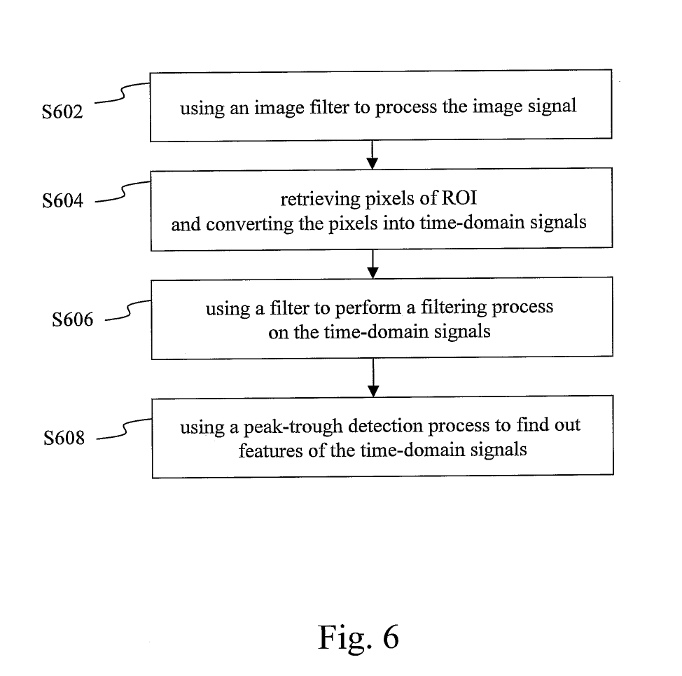

Refer to FIG. 6 for a flowchart of a parametric algorithm of a data processing unit according to one embodiment of the present invention. In Step S602, the parametric algorithm uses an image filter to process the images captured by the image sensing unit 14 and the image analysis unit 18. In Step S604, the pixels of ROI are retrieved and then converted into time-domain signals. In Step S606, a filter performs a filtering process on the time-domain signals. In Step S608, a peak-trough detection process is used to find out the features of the time-domain signals, and the features are used to calculate parameters, such as the reflection index (RI) and the stiffness index (SI).

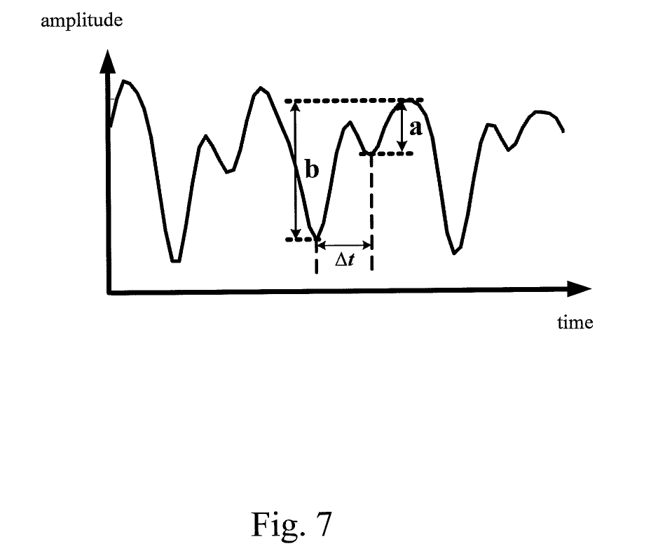

Refer to FIG. 7. RI is defined to be the height of the subject divided by Δt. SI is defined to be the ratio of a to b and expressed by percentage.

Thereby, the user can use the data processing unit to control the image sensing unit 14 and the image analysis unit 18 to capture continuous image signals, and use the software process of the parametric algorithm to calculate the physiological parameters.

Moreover, the conventional respiration measurement technology includes the method of temperature or pressure variation on nostril or mouth and the method of plethysmography on chest. The method of temperature or pressure variation is likely to cause the nose and mouth of the subject to contact the instrument and thus may bring about infection. In the method of plethysmography, the chest belt is likely to loosen, and the subject has to maintain a specified posture during test. Therefore, the two conventional methods respectively have their own drawbacks in practical operation.

Therefore, in another embodiment, the data processing unit 20 of the present invention can work out the physiological parameters of respiration via using the built-in parametric algorithm to perform the respiratory information extraction from the PPG signals.

In one embodiment, the parametric algorithm may use the autoregressive (AR) model for the analysis of PPG signals. AR model is an all-pole model and the transfer function for AR model of order P can be represented as follows.

$$H ( z ) = \frac{{ρ}_{w}}{1 + {\sum }_{k = 1}^{P} {a}_{k} \cdot {Z}^{- k}} = \frac{{ρ}_{w}}{{\prod }_{k = 1}^{P} ( 1 - {p}_{k} \cdot {Z}^{- 1} )} ,$$

where p_{w} denotes the output power of the prediction error,ak's are AR coefficients, whereas pₖ's stand for the poles of the AR model. Each specific pole pₖ (k=1, 2, . . . P) of the AR model can be represented by

pₖ=|pₖ|·e^{j∠p}^{ₖ},

in which |pₖ| and ∠pₖ denote the modulus and argument of pₖ on the complex plane, respectively. The argument ∠pₖ (unit:radian) corresponds to resonant peak in AR spectrum at frequency fₖ (unit:Hz). The relationship between ∠pk and fₖ is

∠pk=2π·fₖ·T,

where T represents the sampling interval (unit:second) of the time-domain PPG signal. The parametric algorithm considers only the dominant pole in the specified frequency range. For example, the respiratory component buried in PPG signal is estimated to be from 0.1 Hz (6 breaths/minute) to 0.4 Hz (24 breaths/minute) in general condition. The respiration frequency is estimated to be the corresponding frequency of the dominant pole in the specified range, and BPM (breaths per minute) is derived according to the equation:

BPM=respiration frequency (Hz)*60 sec

In conclusion, the image-based PWV measurement device and method of the present invention is a PWV measurement technology using a length measurement unit, an image sensing unit and optical elements. The image-based PWV measurement device and method of the present invention can effectively detect PWV without using any expensive instrument.

Further, the present invention uses a data processing unit and the parametric algorithm thereof to analyze the PPG signals, whereby to obtain the physiological parameters, such as the perfusion index, respiration rate, pulse rate, stiffness index, reflection index, and PWV.

The embodiments described above are to demonstrate the technical thoughts and characteristics of the present invention, enabling the persons skilled in the art to understand, make, and use the present invention. However, those embodiments are not intended to limit the scope of the present invention but only to exemplify the present invention. Any equivalent modification or variation according to the spirit of the present invention is to be also included within the scope of the present invention.

What is claimed is:

1. An image-based pulse wave velocity measurement device comprising:
at least two light emitting units respectively projecting light beams to at least two detected regions;
at least two light transmitting units respectively receiving and transmitting light signals measured at the detected regions;
an image sensing unit arranged corresponding to the at least two light transmitting units and converting the light signals measured at the detected regions into image signals;
a length measurement unit, for measuring a distance between the detected regions; and
an image analysis unit connected with the image sensing unit, for analyzing the image signals to obtain two photoplethysmography (PPG) signals for the detected regions, and working out a pulse wave velocity (PWV) between the detected regions according to the distance between the two detected regions and a pulse transit time (PTT) from the two PPG signals.

2. The image-based pulse wave velocity measurement device according to claim 1, wherein the image analysis unit obtains the pulse transit time (PTT) according to the two PPG signals, and wherein the PWV is equal to the distance between the two detected regions divided by the PTT.

3. The image-based pulse wave velocity measurement device according to claim 1, wherein each of the light emitting units includes
a light source module projecting a light beam to one of the detected regions; and
a control module controlling the light source module to project the light beam of different intensities to different detected regions.

4. The image-based pulse wave velocity measurement device according to claim 3, wherein the light source module emits a monochromatic or multi-wavelength light beam, and wherein the light source module is a light emitting diode (LED), a laser diode, or an incandescent lamp.

5. The image-based pulse wave velocity measurement device according to claim 1, wherein each of the light transmitting units is an optical fiber, a reflector, or a refractor.

6. The image-based pulse wave velocity measurement device according to claim 1, wherein the image sensing unit is a CCD (charge coupled device)-based or CMOS (complementary metal oxide semiconductor)-based digital camera device.

7. The image-based pulse wave velocity measurement device according to claim 1 further comprising a data processing unit connected with the image analysis unit and using a parametric algorithm to analyze the two PPG signals to obtain a perfusion index, a respiration rate, a pulse rate, a stiffness index, a reflection index and PWV.

8. The image-based pulse wave velocity measurement device according to claim 7, wherein the data processing unit is a computer, a personal digital assistant, or a mobile phone.

9. An image-based pulse wave velocity measurement method comprising the following steps:
providing at least two detected regions and measuring a distance between the two detected regions;
providing at least two light emitting units respectively projecting light beams to the detected regions;
receiving and transmitting light signals measured at the detected regions, and converting the light signals into image signals;
analyzing the image signals to obtain two photoplethysmography (PPG) signals for the detected regions; and
working out a pulse wave velocity (PWV) between the two detected regions according to the distance between the two detected regions and a pulse transit time (PTT) from the two PPG signals.

10. The image-based pulse wave velocity measurement method according to claim 9 further comprising a step:
obtaining the pulse transit time (PTT) according to the two PPG signals, wherein the PWV is equal to the distance between the two detected regions divided by the PTT.

11. The image-based pulse wave velocity measurement method according to claim 9 further comprising a step:
using a parametric algorithm to perform an image feature analysis and a filtering process on the PPG signals to work out a perfusion index, a respiration rate, a pulse rate, a stiffness index, a reflection index and PWV.

12. The image-based pulse wave velocity measurement method according to claim 11, wherein the parametric algorithm includes the following steps:
using an image filter to process the image signals;
obtaining pixels of a region of interest (ROI) and converting the pixels into time-domain signals;
using a filter to process the time-domain signals; and
using a peak-trough detection process to find out the features of the time-domain signals for calculating the physiological parameters.

13. The image-based pulse wave velocity measurement method according to claim 11, wherein the parametric algorithm includes the following steps:
using an autoregressive (AR) model for the analysis of PPG signals;
finding out a dominant pole in a specified frequency range of respiration according to the autoregressive (AR) coefficients; and
finding out a frequency corresponding to the dominant pole in the specified frequency range of respiration and deriving the respiratory rate per minute.
